(19) United States
(12) Patent Application Publication
(10) Pub. No.: US 2014/0276042 A1
(43) Pub. Date: Sep. 18, 2014

(54) INTEGRATED CATHETER AND POWERED INJECTOR SYSTEM FOR USE IN PERFORMING RADIAL ANGIOGRAPHY

(71) Applicant: SYNTHEON, LLC, MIAMI, FL (US)

(72) Inventors: KEVIN W. SMITH, CORAL GABLES, FL (US); THOMAS O. BALES, JR., CORAL GABLES, FL (US); KOREY KLINE, MIAMI, FL (US); MATTHEW A. PALMER, MIAMI, FL (US); SEAN McBRAYER, MIAMI, FL (US); DEREK DEE DEVILLE, CORAL GABLES, FL (US); ERIC PETERSEN, HOMESTEAD, FL (US)

(73) Assignee: SYNTHEON, LLC, MIAMI, FL (US)

(21) Appl. No.: 14/210,754

(22) Filed: Mar. 14, 2014

Related U.S. Application Data
(60) Provisional application No. 61/787,044, filed on Mar. 15, 2013.
(60) Provisional application No. 61/824,362, filed on May 16, 2013.

(51) Int. Cl. A61M 5/00; A61M 5/145

(52) U.S. Cl. CPC A61M 5/007; A61M 5/14546; A61M 2005/14506; A61M 2202/0007; A61M 2205/8206
USPC 600/433

(57) ABSTRACT

An integrated catheter and powered injector system includes a pump housed in a self-contained unit, a contrast media supply removably connected to the pump by a fluid supply line, an extension line removably connecting an arterial catheter to the pump, and a control device sending user control signals to the pump. The pump comprises a motorized pump drive sub-system operating the pump, and a battery electrically connected to and powering the pump drive sub-system. In response to signals received from the control device, the pump drive sub-system drives the pump to draw contrast media fluid from the supply into the pump through the fluid supply line and inject the contrast media fluid from the output of the pump through the extension line and into the arterial catheter at a controlled flow rate and/or pressure.

### CROSS-REFERENCE TO RELATED APPLICATIONS

This application claims priority to U.S. Provisional Application Ser. No. 61/787,044, filed on Mar. 15, 2013, and 61/824,362, filed on May 16, 2013, the entire disclosures of which are hereby incorporated herein by reference in their entirety.

### STATEMENT REGARDING FEDERALLY SPONSORED RESEARCH OR DEVELOPMENT

Not Applicable

### FIELD OF THE INVENTION

The present invention lies in the field of coronary angiography. In particular, the present invention provides an integrated “one-stop” catheter and powered injector system that is comprised of substantially all of the necessary devices and sub-systems (e.g., a set of catheters of various sizes and geometric configurations and their associated introducer sheaths, a set of angiographic syringes of different designs and sizes, and a battery-powered injector device) needed to perform a coronary angiography procedure through the radial artery of a patient.

### BACKGROUND OF THE INVENTION

Millions of coronary angiography procedures are performed each year in the United States in order to locate blockages in the coronary arteries of the heart and to diagnose patients with coronary artery disease. To perform this procedure, a radiopaque intravenous contrast media (e.g., a fluid dye) is introduced into the bloodstream and its progression through the coronary arteries is monitored by fluoroscopic or other visualizing measures. The size and position of arterial plaque deposits or other arterial occlusions can be visually determined in areas where progression of the contrast media through the arterial network is abnormal. Once these blockages are identified, suitable treatment methods (e.g., balloon angioplasty) for their displacement and/or removal can be performed.

In the majority of cases, the contrast media is introduced into the arterial network through an arterial catheter that has been inserted into the femoral (or iliac) artery, which is a major artery that descends along the anteromedial aspects of the thighs to the junction of the middle and lower third of the thighs. As is well-known in the art, depending on the unique physiology of the patient and which coronary artery (i.e., right or left) is selectively under study, a particular catheter size and/or geometric configuration is used. For example, for imaging the right coronary artery, either a Judkins Right (JR) or an Amplatz Right (AR) catheter may be used. For imaging the left coronary, either a Judkins Left (JL) or an Amplatz Left (AL) catheter may be used. Oftentimes, in coronary angiography procedures that are performed through the femoral (or iliac) artery, the contrast media is injected into the catheter with a manually operated syringe. In a relatively small percentage of cases, the contrast media is introduced instead into the radial artery of the arm through the wrist. Recent studies have determined that angiography performed through the radial artery is just as effective as when performed through the femoral artery. Moreover, when compared to angiography performed through the femoral artery, radial angiography is reported to have fewer associated complications, is generally a more comfortable procedure for the patient, causes less bleeding, and has a faster recovery time.

Still, there are several disadvantages associated with radial angiography, as well. For example, the radial artery is a small branch of the brachial artery that is found in the arm and wrist. As such, the radial artery has a progressively smaller-sized diameter when compared to the femoral artery that is found in the thigh or groin area. Accordingly, it is difficult to access the radial artery with a catheter and to inject the contrast media at a pressure and flow rate that approximates or falls within the pressure tolerances of the radial artery. Entry into the radial artery is even more difficult for women as the radial artery is even smaller, generally speaking, in women than in men. Therefore, a small-sized catheter having a relatively tiny diameter must be used. For example, a 4-French catheter is typically chosen to perform a radial angiography. In fact, in view of these considerations, specialized catheters have been invented for particular use in performing radial angiography. Yet, by using the appropriate catheter, it then becomes impossible to manually inject the contrast media into such a tiny diameter and at a controlled (or steady) rate and pressure. Complications exist either way. If a catheter having too large of a diameter is used or, if the contrast media is injected into smaller catheter at a rate or pressure that is too high, a number of complications can occur. Examples of the more dangerous complications include extensive hemorrhaging, damage or paralysis of the nerves surrounding the artery, and the formation of a blood clot (thrombus) in the artery. Accordingly, in a radial angiography procedure, injection of the contrast media into the catheter must be performed under a controlled flow rate and pressure using a powered injector device.

A powered injector is a device that injects fluids into a patient at a controlled rate or pressure. Typically, to perform a coronary angiography procedure, the powered injector is manually loaded with a sterile angiographic syringe that is either empty or pre-filled with the contrast media by the syringe manufacturer. In most cases, the powered injector has an automated pump that advances the plunger of the loaded angiographic syringe under the control of, for example, a microprocessor that is programmed to control certain injection parameters of the syringe. Examples of such parameters may include the flow rate, flow volume, and flow pressure. When the powered injector is loaded with an empty syringe, the desired amount of contrast media needed for the injection is drawn into the syringe nozzle from a nearby supply using, for example, the automated pump of the powered injector to draw the plunger of the syringe backwards.

There is a variety of designs and styles of both the pre-filled and empty angiographic syringes. Which syringe type is used is oftentimes the particular choice of the physician or may be dictated by the equipment of the powered injector that is being used.

With the introduction of a powered injector device, there is an added operational complexity and expense because an entire system must be put into place in order to incorporate the powered injector device.

It would be beneficial to reduce or eliminate the added complexity and expense of incorporating a traditional powered injector system that is set up using a building block approach by combining one or more components of this added system into several integrated systems that are easily replaceable, are quickly connectable to one another, and are powered by a self-contained power source.

Accordingly, a need exists to overcome the problems with the prior art systems, designs, and processes that use a powered injector device for performing radial angiography procedures.

### SUMMARY OF THE INVENTION

The present invention provides a simplified, more-easily used, more compact, and integrated injection system that can be operated under the physician's direct control. By providing an “all-in-one” system, the present invention provides a powered injector system that is simpler in comparison to the prior art systems because, for example, it requires fewer connections between its component parts, eliminates cords, and eliminates the capital equipment of a traditional powered injector.

With the foregoing and other objects in view, there is provided, in accordance with the invention, a n integrated catheter and powered injector system, including a pump system housed in a self-contained unit, a contrast media supply containing contrast media fluid, an extension line, and a control device. The pump system comprises a motorized pump drive sub-system, a pump electrically connected to and operated by the pump drive sub-system, the pump having a input and an output, and a battery electrically connected to and powering the pump drive sub-system. The contrast media supply is removably connected to the pump system by at least one fluid supply line and contains the contrast media fluid. An extension line is shaped to removably connect an arterial catheter to the pump system. A control device is operatively connected to the pump system to allow a user to send control signals to the pump system such that, in response to control signals received from the control device, the pump drive sub-system drives the pump to draw the contrast media fluid from the contrast media supply into the input of the pump through the at least one fluid supply line and inject the contrast media fluid from the output of the pump through the extension line and into the arterial catheter at a controlled flow rate and/or pressure.

In accordance with another feature of the invention, the battery is removable.

With the objects of the invention in view, there is also provided an integrated catheter and powered injector system comprising a battery-powered pump drive system, a peristaltic pump electrically connected to and operated by the pump drive system, the peristaltic pump having an input and an output, a removable battery electrically connected to and powering the pump drive system, a contrast media supply removably connected to the input of the peristaltic pump by at least one fluid supply line, an extension line shaped to removably connect an arterial catheter to the output of the peristaltic pump, and a control device operatively connected to the pump drive system to allow a user to send control signals to the pump drive system such that, in response to control signals received from the control device, the pump drive system drives the peristaltic pump to draw contrast media fluid from the contrast media supply into the input of the peristaltic pump through the at least one fluid supply line and inject the contrast media fluid from the output of the peristaltic pump through the extension line and into the arterial catheter at a controlled flow rate and/or pressure.

Although the invention is illustrated and described herein as embodied in an integrated catheter and powered injector system for use in performing radial angiography, it is, nevertheless, not intended to be limited to the details shown because various modifications and structural changes may be made therein without departing from the spirit of the invention and within the scope and range of equivalents of the claims. Additionally, well-known elements of exemplary embodiments of the invention will not be described in detail or will be omitted so as not to obscure the relevant details of the invention.

Additional advantages and other features characteristic of the present invention will be set forth in the detailed description that follows and may be apparent from the detailed description or may be learned by practice of exemplary embodiments of the invention. Still other advantages of the invention may be realized by any of the instrumentalities, methods, or combinations particularly pointed out in the claims.

Other features that are considered as characteristic for the invention are set forth in the appended claims. As required, detailed embodiments of the present invention are disclosed herein; however, it is to be understood that the disclosed embodiments are merely exemplary of the invention, which can be embodied in various forms. Therefore, specific structural and functional details disclosed herein are not to be interpreted as limiting, but merely as a basis for the claims and as a representative basis for teaching one of ordinary skill in the art to variously employ the present invention in virtually any appropriately detailed structure. Further, the terms and phrases used herein are not intended to be limiting; but rather, to provide an understandable description of the invention. While the specification concludes with claims defining the features of the invention that are regarded as novel, it is believed that the invention will be better understood from a consideration of the following description in conjunction with the drawing figures, in which like reference numerals are carried forward.

### BRIEF DESCRIPTION OF THE DRAWINGS

The accompanying figures, where like reference numerals refer to identical or functionally similar elements throughout the separate views, which may not be true to scale, and which, together with the detailed description below, are incorporated in and form part of the specification, serve to further illustrate various embodiments and to explain various principles and advantages all in accordance with the present invention. Advantages of embodiments of the present invention will be apparent from the following detailed description of the exemplary embodiments thereof, which description should be considered in conjunction with the accompanying drawings in which:

FIG. 1 is a system diagram of a first exemplary embodiment of an integrated catheter and powered injector system according to the invention;

FIG. 2 is a system diagram of a second exemplary embodiment of an integrated catheter and powered injector system according to the invention;

FIG. 3 is a system diagram of a third exemplary embodiment of an integrated catheter and powered injector system according to the invention;

FIG. 4 is a system diagram of a fourth exemplary embodiment of an integrated catheter and powered injector system according to the invention;

FIGS. 5(a), 5(b), and 5(c) are fragmentary, cross-sectional views of tubing segments of a flexible tube of a peristaltic pump according to the invention;

FIG. 6 is a fragmentary system diagram of a gear pump and pump drive system portion of a further exemplary embodiment of an integrated catheter and powered injector system according to the invention;

FIG. 7 is a photograph of an exemplary embodiment of a media control pump according to the invention with a top cover separated from a main body thereof;

FIG. 8 is a perspective view of an exemplary embodiment of a media control pump according to the invention with a top cover separated from a main body thereof and with a syringe and piston transparent;

FIG. 9 is a perspective view of the media control pump of FIG. 8 with the syringe removed;

FIG. 10 is a fragmentary, enlarged perspective view from a left side of the media control pump of FIG. 8 with the syringe and piston removed;

FIG. 11 is a fragmentary,enlarged perspective view from a right side of the media control pump of FIG. 8 with the syringe and piston removed;

FIG. 12 is a fragmentary,enlarged perspective view from a below the media control pump of FIG. 8 with the top cover present and a bottom body half removed;

FIG. 13 is a perspective view of a lower right side of the piston of FIG. 8;

FIG. 14 is a perspective view of a lower left side of the piston of FIG. 8;

FIG. 15 is a perspective view of the media control pump of FIG. 8 with the top cover present;

FIG. 16 is a perspective view from a distal end of the media control pump of FIG. 8;

FIG. 17 is a fragmentary, perspective view of a proximal end of the media control pump of FIG. 8 with the piston transparent;

FIG. 18 is a fragmentary, hidden line perspective view of the proximal end of the media control pump of FIG. 8;

FIG. 19 is an enlarged perspective view of a proximal end of the media control pump of FIG. 8 with the piston and an end cap removed;

FIG. 20 is an enlarged perspective view of a distal end of the media control pump of FIG. 8 with a circuit board;

FIG. 21 is an enlarged perspective view of a distal end of the media control pump of FIG. 8 with a speed control device;

FIG. 22 is a perspective view of an exemplary embodiment of a remote hand control device according to the invention connected to an entry sheath;

FIG. 23 is a perspective view of the remote hand control device of FIG. 22;

FIG. 24 is a fragmentary, enlarged, perspective view of the remote hand control device of FIG. 22;

FIG. 25 is an enlarged perspective rear view of the two-stage switch of the remote hand control device of FIG. 22;

FIG. 26 is a perspective front view of the two-stage switch of the remote hand control device of FIG. 22 with the two-stage switch removed;

FIG. 27 is a perspective rear view of another exemplary embodiment of a remote hand control device according to the invention;

FIG. 28 is a perspective rear view of the remote hand control device of FIG. 27;

FIG. 29 is a perspective front view of the remote hand control device of FIG. 27;

FIG. 30 is a perspective rear view of still another exemplary embodiment of a remote hand control device according to the invention;

FIG. 31 is a side elevational view of the remote hand control device of FIG. 30;

FIG. 32 is a perspective front view of the remote hand control device of FIG. 30; and

FIG. 33 is a perspective rear view of the remote hand control device of FIG. 30.

### DETAILED DESCRIPTION OF THE INVENTION

As required, detailed embodiments of the present invention are disclosed herein; however, it is to be understood that the disclosed embodiments are merely exemplary of the invention, which can be embodied in various forms. Therefore, specific structural and functional details disclosed herein are not to be interpreted as limiting, but merely as a basis for any claims and as a representative basis for teaching one skilled in the art to variously employ the present invention in virtually any appropriately detailed structure. Further, the terms and phrases used herein are not intended to be limiting; but rather, to provide an understandable description of the invention. While the specification may conclude with claims defining the features of the invention that are regarded as novel, it is believed that the invention will be better understood from a consideration of the following description in conjunction with the drawing figures, in which like reference numerals are carried forward.

Before the present invention is disclosed and described, it is to be understood that the terminology used herein is for the purpose of describing particular embodiments only and is not intended to be limiting. The terms “a” or “an”, as used herein, are defined as one or more than one. The term “plurality”, as used herein, is defined as two or more than two. The term “another”, as used herein, is defined as at least a second or more. The terms “including” and/or “having”, as used herein, are defined as comprising (i.e., open language). The term “coupled,” as used herein, is defined as connected, although not necessarily directly, and not necessarily mechanically. Relational terms such as first and second, top and bottom, and the like may be used solely to distinguish one entity or action from another entity or action without necessarily requiring or implying any actual such relationship or order between such entities or actions. The terms “comprises,” “comprising,” or any other variation thereof are intended to cover a non-exclusive inclusion, such that a process, method, article, or apparatus that comprises a list of elements does not include only those elements but may include other elements not expressly listed or inherent to such process, method, article, or apparatus. An element proceeded by “comprises . . . a” does not, without more constraints, preclude the existence of additional identical elements in the process, method, article, or apparatus that comprises the element.

As used herein, the term “about” or “approximately” applies to all numeric values, whether or not explicitly indicated. These terms generally refer to a range of numbers that one of skill in the art would consider equivalent to the recited values (i.e., having the same function or result). In many instances these terms may include numbers that are rounded to the nearest significant figure.

It will be appreciated that embodiments of the invention described herein may be comprised of one or more conventional processors and unique stored program instructions that control the one or more processors to implement, in conjunction with certain non-processor circuits and other elements, some, most, or all of the functions of the powered injector devices described herein. The non-processor circuits may include, but are not limited to, signal drivers, clock circuits, power source circuits, and user input and output elements. Alternatively, some or all functions could be implemented by a state machine that has no stored program instructions, or in one or more application specific integrated circuits (ASICs) or field-programmable gate arrays (FPGA), in which each function or some combinations of certain of the functions are implemented as custom logic. Of course, a combination of these approaches could also be used. Thus, methods and means for these functions have been described herein.

The terms “program,” “software application,” and the like as used herein, are defined as a sequence of instructions designed for execution on a computer system. A “program,” “computer program,” or “software application” may include a subroutine, a function, a procedure, an object method, an object implementation, an executable application, an applet, a servlet, a source code, an object code, a shared library/dynamic load library and/or other sequence of instructions designed for execution on a computer system.

Described now are exemplary embodiments of the present invention.

Referring now to the figures of the drawings in detail and first, to FIG. 1, there is shown a diagrammatic, general overview of a first exemplary embodiment of an integrated catheter and powered injector system 10 according to the invention for use in performing a radial angiography procedure. Even though this exemplary embodiment is illustrated as a system for use in performing a radial angiography procedure, this embodiment is not to be considered as limited thereto. The integrated catheter and powered injector system 10 disclosed herein may be used in any diagnostic angiography procedure that uses a powered injector device.

In FIG. 1, various principle devices and sub-systems of the integrated catheter and powered injector system 10 are shown in a first assembled configuration. The components of the system 10 shown in FIG. 1 include a power source 20, a contrast media supply 30, an automated pump 40 and its associated pump drive system 50, an arterial catheter 70, and a hand control device 80. In this particular exemplary embodiment, only the components (e.g., catheter 70 and hand control device 80) of the system 10 that are manipulated by the physician, or come into contact with the patient during the angiography procedure, are made sterile and are maintained in a sterile area 100. All other components of the system do not have to be made sterile and can be positioned in a non-sterile environment 90 that is outside of the sterile area 100. In FIG. 1, the demarcation between the non-sterile 90 and sterile 100 areas is shown as a dashed line 1. These key components of the system 10 are easily interconnected using a simple network of fluid and electrical supply lines 15, 25, 35, 45, 55.

Any suitable power source 20 may be used for powering the system 10. In FIG. 1, the power source 20 is a battery.

In this particular exemplary embodiment, the automated pump system 110 of the powered injector device 130 is comprised of a peristaltic pump 40 and an electrical pump drive system 50 that is operatively connected thereto. However, as described in detail below, a peristaltic pump 40 is just one of several types of types of pumps that can be used in the present invention. The pump 40 is operated by the pump drive system 50, which is controlled by the user with a sterile (remote) hand control device 80 that is connected to the pump drive system 50 by an electrical control cable 55. The pump drive system 50 is comprised of at least one drive motor (not shown) that is electrically coupled to at least one drive control circuit (not shown). In some exemplary embodiments, the at least one drive control circuit may be comprised of at least one microprocessor (not shown). The pump drive system 50 is supplied with electrical power from the power source 20 through an electrical wire or cable 25. When the pump 40 is being driven by the pump drive system 50, contrast media fluid is drawn into the pump 40 through a fluid supply or media access line 15 that is connected to the contrast media supply 30. The contrast media supply 30 may be comprised of an iodinated contrast media fluid that is housed in a specific container (e.g., in a glass bottle with connection as by needle or bowl). To accomplish this filling process, the fluid supply line 45 is attached to a three-way valve or manifold (not shown) of an integrated check valve filling system of the pump system 110. Depending on which type of pump is being used, the contrast media may be drawn into an empty angiographic syringe or a pump cartridge 60 that has been pre-loaded or mounted to the pump system 110. In this exemplary embodiment, the fluid and electrical supply lines 15 and 25, respectively, that lead out from the contrast media supply 30 and the power source 20 are advantageously combined or are purposefully intertwined into a single cable 35 for the majority of the distance separating the contrast media supply 30 and the power source 20 from the pump system 110. Just prior to entering their separate components 40, 50 of the pump system 110, the cable 35 splits again into its constituent fluid and electrical supply lines 15, 25.

Referring now to FIG. 2, the devices and sub-systems of the integrated catheter and powered injector system 10 are shown in a second exemplary configuration. Similar to FIG. 1, the demarcation between the non-sterile 90 and sterile 100 areas is shown as a dashed line 1. In this second configuration, a pump cartridge 160 of the peristaltic pump 140 is made surgically sterile and is positioned in the sterile area 100 along with the sterile arterial catheter 70 and the sterile hand control device 80. For purposes of the present invention, a “pump cartridge” is comprised of a housing that might contain mechanical connection measures, a pump of some sort (e.g., peristaltic, piston, etc.), sensors (such as pressure, torque, position, and temperature), and measures for connecting fluid input and output lines to the pump. The pump cartridge may also include the pump tubing or it may have measures for connecting the tubing lines. The pump drive system 50 remains in the designated non-sterile area 90 and is connected to the pump 140 through one or more electrical lines 145. Electric power from the power source 20 is supplied to the pump drive system 50 using an electrical line 135. The pump 140 draws contrast media from the contrast media supply 30 and into the pump cartridge 160 through the fluid supply or media access line 125.

Illustrated in FIG. 3 is a third exemplary configuration of the devices and sub-systems of the integrated catheter and powered injector system 10. In this configuration, a battery 120 has been integrated with a pump cartridge 260 of the peristaltic pump 240 to form a single assembly unit. As a result, the entire battery and pump cartridge assembly 120-240-260 can easily be made sterile and maintained in the sterile area 100 along with the arterial catheter 70 and hand control device 80. The pump drive system 50 remains in the designated non-sterile area 90 and is connected to the pump 240 using at least one electrical line 195 that traverses the sterile/non-sterile barrier 1. The pump drive system 50 is supplied with electrical power from the battery 120 by an electrical cable 185 that also traverses the sterile/non-sterile barrier 1. The pump 240 draws contrast media from the contrast media supply 30 and into the pump cartridge 260 through the fluid supply or media access line 175.

Referring now to FIG. 4, there is shown a fourth exemplary configuration of the devices and sub-systems of the integrated catheter and powered injector system 10. In this configuration, a battery 220, a pump 340, and the pump drive system 250 are all integrated into a single, self-contained assembly or unit 200, thereby advantageously eliminating the intervening electrical cables of the embodiments of FIGS. 1, 2, and 3 that connect the power source, the pump drive system, and the pump. As a result, in comparison to the embodiments of FIGS. 1, 2, and 3, in which one or more of the power source, pump drive system, and pump are physically separate and apart from each other, this single assembly or unit 200 of the embodiment of FIG. 4 increases the portability and simplicity of the system 10. The unit 200 is positioned in the designated non-sterile area 90. The pump 340 draws contrast media from the contrast media supply 30 and into the pump 260 through the fluid supply or media access line 205.

In each of the embodiments described above, a proximal end of an extension hose or tube (shown as parts 65, 105, 165, and 215 in FIGS. 1 to 4, respectively) is coupled to the pump 40, 140, 240, 340 or, more specifically, to the pump cartridge or angiographic syringe 60, 160, 260, depending on the type of pump that is used. The opposite distal end of the extension hose or tube 65, 105, 165, 215 is coupled to the arterial catheter 70. Upon use of the hand control device 80, the pump 40, 140, 240, 340 injects the contrast media into the catheter 70 and through to the patient through the extension hose or tube 65, 105, 165, 215. The hand control device 80 may comprise a pad having a number of buttons or switch controls. Alternatively, the hand control device 80 may have the appearance and feel and mimic the operation of an actual, manually operated angiographic syringe. In this way, a physician (or other practitioner) who is accustomed to using an actual syringe and tp performing manual injections by the femoral route, will still have the similar feel of an actual syringe when he or she is using the hand control device 80.

Having now been given a general overview of the exemplary embodiments of the integrated catheter and power injector system 10, below is a detailed description of certain inventive aspects of the various devices and sub-systems that comprise the system 10.

FIGS. 1 to 4 illustrate just four non-limiting examples of the assembled configurations (or arrangements) of the integrated catheter and powered injector system 10. There are a variety of possible combinations and configurations of the devices and sub-systems of the system 10 that are contemplated. Importantly, irrespective of the chosen configuration of the integrated catheter and powered injector system 10, the devices and sub-systems thereof are to be easily assembled together using a number of either specialized or universal fittings and/or connectors.

For example, as described above, in circumstances in which an empty angiographic syringe or pump cartridge 60, 160, 260 has been loaded onto or mounted to the pump 40, 140, 240, 340, the contrast media fluid is supplied from the contrast media supply 30 to the syringe or pump cartridge through the fluid supply or media access line 15, 125, 175, 205. As also described above, a proximal end of an extension hose or tube 65, 105, 165, 205 is subsequently coupled to a discharge section of the syringe or pump 60, 160, 260, and the distal end of the extension hose or tube 65, 105, 165, 205 is attached to the catheter 70, thereby providing a fluid pathway between the powered injector and the patient. During the process of connecting each of these fluid supply lines to their devices, air may be inadvertently introduced into the tubing of the pump 40 or into the extension hose or tube 65, 105, 165, 205 and, therefore, upon injection into the catheter 70, an air embolism may form in the patient's bloodstream with dangerous consequences. Accordingly, to prevent any air bubbles from entering the catheter 70, one or more double shut-off valves or connectors (not shown) may be configured for use at either the proximal side (i.e., power injector side) of the extension hose or tube 65, 105, 165, 215 or at the distal side (i.e., catheter/patient side) of the extension hose or tube 65, 105, 165, 215.

Also, on either side of the pump system 110, a number of alternative connector configurations can be use for fast and easy assembly. In one example, one or more non-shutoff connectors may be used. In this type of connector, no valves are employed but, rather, the connectors are open to the air when disconnected. In another example, two-position connectors can be used that mate in either a connected or a shutoff configuration. In a further example, a single-shutoff connector system may be used, whereby a valve is used on the proximal side (i.e., contrast media supply side) of the pump system 110 to prevent ingress of air into the lines or pump(s). Or, alternatively, a valve may be used on the distal side (i.e., catheter/patient side) of the pump system 110 to prevent backflow of blood when disconnected. In an alternative example, a double-disconnect connector system may be used in which a valve is used on both the proximal side and distal side of the pump system 110, the valves being opened only when the connectors on both sides are mated, thereby preventing ingress or egress of fluids. In another example, a valve and weep system may be used. In such a system, one or both sides of the pump system 110 can be provided with a valve that, when disconnected, has a weeping feature that still allows a small flow such that, if used on the proximal side (i.e., contrast media supply side) of the pump, it will allow bleeding of air without disconnecting the fluid supply or media access line 45, 125, 175, 205. If used on the distal side (i.e., catheter/patient side), the weeping feature will allow a suction extraction of fluids without allowing free blood egress when disconnected. This type of valve would only open at pressures above normal blood pressure ranges. Lastly, in addition, air-permeable membranes or filters could be used at either the inlet or outlet side of the pump to allow purging of air without having to actuate the valve.

With respect to any of the mechanical connectors used in the system 10, such connectors can be of any suitable type, including bayonet, twist-lok, screw, magnetic, or snap (either lever-actuated or axial-press-fit) connectors. The connectors can be hermaphroditic to allow flexibility in how the connection can be made. Hermaphroditic connectors can be of the bayonet, magnetic, or interlocking threads, etc. type. In addition, the connectors can have a swivel capability, especially at the catheter, to allow for easy manipulation at the connection.

With respect to the powered injector device 130, as illustrated in the exemplary embodiment of FIG. 4, the device 130 may be comprised of a pump, a pump drive system, and a battery that are all combined or integrated into a single, self-contained unit. By combining or integrating all three of these components into a single unit, that unit may be made to be relatively compact and lightweight, such that it is portable and can be easily transported into an area adjacent to or shielded from the sterile field. Additionally, some or all of the components of this integrated powered injector device 30 could be configured as disposable or reusable for their easy replacement. Disposable components may be discarded after a certain amount of use and replaced with sterile replacements. Reusable components can be removed, sterilized, and reused in a future procedure. In fact, because this integrated powered injector is now self-contained in a single unit, the entire device 130 may be easily “bagged” after sterilization, such that it never comes into contact with and is not contaminated by its surrounding environment. The only external devices that need to be connected to this single unit are the catheter 70, the hand control device 80, and the contrast media supply 30 using the fluid supply or media access line 45, 125, 175, 205, the extension hose or tube 65, 105, 165, 215, the electrical control cable 55, 115, 155, 225, and the “quick-connect” fittings and connectors needed to provide the necessary connections. In one exemplary embodiment, the fluid supply line 45, 125, 175, 205 and the three-way or check valve connection for connecting the fluid supply line to the pump system 110 may be provided as one integrated filling system. Therefore, this integrated filling system and the extension hose or tube 65, 105, 165, 215 may be easily mated to the reusable, self-contained powered injector device 130 described just above. With such a configuration, all components that are wetted or in contact with the physician or patient could be replaced as one unit when necessary or desired.

Alternatively, in another exemplary embodiment, the integrated filling system may be integrated into the self-contained powered injector device 130 and only the extension hose or tube 65, 105, 165, 215 would need to be connected to the injector system 130.

To even further simplify the entire system 10, one or more replaceable, pre-filled angiographic syringes or pump cartridges could be used with, or integrated into, the self-contained powered injector device 130, thereby eliminating the need to have a contrast media supply 30 and the supply line available for filling empty syringes or cartridges. By using replaceable syringes or pump cartridges, it becomes possible to limit use of that particular syringe or pump cartridge to, for example, just one day or one patient, before it needs to be discarded or replaced. This positive control of discarding and replacing the syringes or pump cartridges at specific points in time is still made possible even if the replaceable angiographic syringes or pump cartridges are provided as empty. This system configuration allows for a quick replacement of all of the wetted components.

Turning to the actual pump 40, 140, 240, 340, it has thus far been identified and illustrated in FIGS. 1 to 4 as being a peristaltic pump. Specific features of peristaltic pumps make them highly suitable for use in powered injector devices used for the purpose of performing coronary angiography. In general, a peristaltic pump is comprised of an outer housing, a flexible tube configured to allow fluid to flow therethrough, and one or more rotating rollers that are supported by the outer housing. Each roller, as it rotates, moves along the flexible tube and exerts a pressure against the tube causing any fluid inside that part of the tube to be forcibly propelled forward through the tube. As the roller continuous to rotate and move along the flexible tube, the previously compressed part of the tube regains its original shape causing a negative pressure inside the tube. This resulting vacuum draws more fluid into the tube from an external fluid source such that fluid is continually entering and traveling through the tube. Through this repetitive operation of the rotating rollers, a pumping action is created. Any backflow of the fluid into the tube is also prevented by having multiple rollers. Because the fluid is encased inside the tube at all times, it never comes into contact with the mechanical components of the pump and is never exposed to the outside environment. Therefore, a peristaltic pump is oftentimes used to transport and pump sterile fluids. Accordingly, a peristaltic pump is a logical choice for use in the powered injector device of the present invention. However, there are a number of appropriate alternative pump types that may be used instead of a peristaltic pump to provide a controlled injection of the contrast media fluid.

For example, a mechanized syringe pump can be used for the pump 40, 140, 240, 340. In such a pump, one or more sterile angiographic syringes are loaded into or mounted on the syringe pump. In general, the piston (or plunger) of the syringe is connected or coupled to a pushing element of the pump. The pushing element is coupled or fastened to a motorized drive element of the pump. Upon controlled actuation of a drive motor of the pump, the pushing element causes the piston of the syringe to be pulled or pushed within the cylindrical barrel of the syringe. When the piston is pulled, a negative pressure is created in the syringe barrel thereby drawing fluid into the barrel. When the piston is pushed, a positive pressure is created in the syringe barrel causing the fluid to be forcibly dispensed out from the barrel. Here, there are several ways in which a syringe pump could be made to be more suitable for the high-pressure injections needed to perform radial angiography procedures. The syringe used could be a single use (disposable), high-strength syringe that will support the high pressure itself. The syringe (and piston) could be comprised of a reinforced housing to prevent leaks if the syringe barrel expands under the pressure. In addition, the piston may be fitted with a compliant seal (e.g., an O-ring) that can accommodate any expansion of the barrel that might occur.

In another example, a gear pump can be used for the pump 40, 140, 240, 340 in, for example, any of the four configurations shown in FIGS. 1 to 4. Due to their rigid components, gear pumps are known in the art as being useful for pumping fluids at high pressures where a constant amount of fluid is pumped per gear-revolution. Possible types include, but are not limited to, twin-gear, single-gear and crescent, gerotor, and roots-type rotary pumps. When using a gear pump, it may be beneficial to place a filter in line with the catheter to prevent any particulates from entering the patient's bloodstream and causing an embolism. Also, a check valve or a valve that shuts at low pressures may be necessary to avoid backflow of blood or other fluids in contact with the patient into the gear pump. The gear pump could be used with a disposable pump cartridge. In addition, as generally describe above with regard to all pump types, the tubing necessary to connect the pump to the contrast media supply and to the catheter may be provided as a disposable set and may be integrated with, for example, the gear pump. In FIG. 6, there is shown one exemplary embodiment of the pump system 110 of a powered injector device 30 where the pump 500 is a gear pump. The gear pump 500 is comprised of a pump cartridge 540 and a pump drive system 550, the pump cartridge being positioned on the sterile side 100 of the sterile/non-sterile barrier 1 and the pump drive system 550 being positioned on the non-sterile side 90 of the barrier 1. Thus, in this exemplary embodiment, to drive the pump, a first side 510 of a magnetic drive module/armature 510 is positioned at the pump cartridge 540 and a second side 520 of the magnetic drive is positioned at the pump drive system 550 adjacent to and substantially aligned with the first side of the magnetic drive.

In a further example, a piston pump or reciprocating pump can be used for the pump 40, 140, 240, 340. The small stroke of the piston of a reciprocating pump is beneficial because it provides a small displacement of fluid and a quick refill, thereby providing a nearly continuous flow. Multiple cylinders may be used to create a more uniform flow. In addition, the small-displacement piston(s) reduce the amount of contrast media fluid that may otherwise be wasted at the end of a case.

If a peristaltic pump is employed as the pump 40, 140, 240, 340, the following considerations may be made to improve its use for the above-described surgical application. For example, if a sterile pump cartridge is used in connection with the non-sterile pump drive system of the peristaltic pump, the pump cartridge could be contained within an impervious bag, or other type of barrier device, and coupled to the pump drive system through the bag (or other device) without compromising the sterile environment inside the bag (or other device). The flexible pump tubing could penetrate the barrier device and the areas of penetration may be sealed to prevent contamination from entering through those areas. As a result, the pump cartridge remains sterile throughout its use. In another example, to reduce flow pulsation in the output of the pump, the pump may be configured to have offset or non-synchronous flow from two or more parallel channels to provide a two-phase or multi-phase system. In addition, the pump could use a “harmonic drive” to achieve a high gear-reduction in a small space.

A number of additional considerations can be made regarding the characteristics of the flexible tubing that is used in the peristaltic pump. For example, depending upon the resistance and elasticity of the inlet tube to the pump, the inlet tube may limit the desired speed of the pump. Accordingly, the inlet tube should have a large enough diameter and a good elastic recovery to take up the contrast media fluid with ease and with a sufficient amount of suction. Nitinol (NiTi) tubing is a prime example of a tubing material having super elasticity and shape memory. In the area of the tubing that is compressed or “pinched” by the one or more rollers of the pump (i.e., in the area of the tube subject to the peristaltic motion), the tubing must have a virtually perfect seal so that there will not be any backflow resulting in reduced flow and pressure. And, in the high pressure side of the tubing, it is especially important that the tubing is comprised of a material that resists high pressure. Accordingly, in FIGS. 5(a), 5(b), and 5(c), there are shown examples of how certain areas or segments of the same flexible tubing may be specially reinforced depending on where that particular area or section of the tube resides in the pump. As shown in FIG. 5(a), the tubing wall 400 of the low pressure areas of the tubing may be comprised of, for example, only a solid polymer material such as silicone, urethane, nylon, or an alloy or co/polymer. As shown in FIG. 5(b), in the high pressure areas of the tubing, the tubing wall 400 may be comprised of a polymer that has been internally reinforced using, for example, braided, coiled, or linear wires 410. However, as shown in FIG. 5(c), in the areas of the tubing that are subject to the peristaltic motion, the tubing wall may be comprised of, for example, a reinforced Nitinol tubing 420 having a polymer lining 400.

Control and operation of the pump 40, 140, 240, 340 and the pump drive system 50, 250 of the powered injector device 130 may range in level of complexity from one embodiment to another. In one exemplary embodiment, the control and operation of the device 130 may be accomplished using an electronic control system that uses only hardware electronics and is free of any software components (including a microprocessor). In such an embodiment, the switches, motors, solid state motor drivers, and redundant safety stop controls, along with the battery, can be used. Thumb-force-controlled injection may be employed. A pressure switch cut-out can be used to limit the flow pressure of the pump. Additionally, this electric-only control system may have feedback control for smooth limiting of the pump pressure using the thumb pressure for modulation. Furthermore, this control system can provide various feedback indicators to the operator. These indicators may indicate, for example, the occurrence of a pressure limit condition, a near-end-of-cartridge condition, or an inadequate (or low) battery charge. These indicators may comprise lights, buzzers, sounders, a display screen, and/or tactile (haptic) elements. These indicators may be located in the hand control device by, for example, integrating the indicators into the buttons themselves. These indicators may be located anywhere in the system as long as the user can see, hear, and/or feel them as necessary.

Alternatively, to allow for higher-complexity functions, at least one programmable microcontroller and associated software may be incorporated into the control and operation of the pump 40, 140, 240, 340 and the pump drive system 50, 250 of the powered injector device 130. Such higher functions may include, for example, electronic interrogation of the pump cartridge, the catheter, or any other component of the system 10 to authenticate its identity, to determine its size, pressure limit, or fluid properties, and/or to obtain patient-safety data (particularly in cases where an ionic contrast media is used). Feedback control of the powered injector device 130 of a motor drive of the pump drive sub-system 50 may be accomplished using a PID controller or Kalman control and by using one or more sensed parameters (e.g., thumb pressure, fluid pressure, piston force, travel distance, temperature, and/or flow rate using a change-in-pressure (AP) sensor) as input into the feedback control system. Any suitable microcontroller may be used. However, radiation-hardened microcontroller chips with a fusible-link memory or laser-programmed, mask-programmed, or shielded gamma-resistant microcontrollers are the best suited for this particular use or application so that the chips can withstand sterilization processes that use gamma rays. With regard to logic type, the one or more microcontrollers may be of a FPGA, CPLD, ASIC, or SoC architecture type.

As described above and as pictured in FIGS. 1 to 4, the pump drive system 50, 250 is controlled by the user with a remote hand control device 80. As a result, the hand control device 80 must always be positioned in the sterile field and be easily accessible by the user (e.g., physician). Despite this limitation, the hand control device 80 may be integrated into a number of the components of the system 10. For example, the hand control device 80 may be integrated with the pump drive system 50, 250, the pump cartridge (peristaltic, syringe, or gear) 60, 160, 260, or the extension hose or tube 65, 105, 165, 215 that leads to the catheter 70. If the hand control device 80 is integrated with the pump drive system 50, 250 in any system configuration that places the pump drive system outside of the sterile field, the hand control device 80 may traverse the sterile barrier by, for example, an electrical control cable. The hand control device 80 may also have its own extension wire to increase the user's mobility. In another exemplary embodiment, the control device 80 may, instead, be in the form of a foot-control device, which frees the user's hands from having to operate the control device. The hand controls may be advantageously located near the site where the system goes into the patient's arm because that is where the physician is directing his or her attention. To better facilitate this configuration, the hand control device 80 may be made entirely wireless so that it may be used from any distance in which a wireless signal may reliably travel in that particular environment. Any suitable type of wireless technology may be used. For example, near-field RF is well-suited for this application because of its low power requirements. Or, the control signals could be conducted by a conductive element, such as a wire reinforcement, in the catheter and/or the extension hose or tube connected to the catheter. Another way to transmit the control signals is by ultrasonic sound waves or by infrared signals through the air.

Turning now to the battery component of the system 10, the battery 20, 120, 220 may be strategically integrated into various components of the system 10, thereby eliminating the system of a separate battery component. Furthermore, the size (i.e., energy capacity) of the battery may be specifically determined by the component into which it is integrated. More specifically, for example, the battery 20, 120, 220 could be integrated into the cylinder of the disposable angiograph syringe or into the pump cartridge 60, 160, 260 and the necessary battery capacity may be determined by the calculated amount of energy that will be required to deliver the volume of the syringe or the pump cartridge either once (if pre-filled), or several times consistent with one procedure. In another example, the battery 20, 120, 220 could be integrated into the contrast media container 30 so that the necessary battery capacity can be determined by the calculated amount of energy that is required to inject the volume of media contained in the container at a maximum pressure. As shown in the exemplary embodiment of FIG. 1, the battery 20 has been integrated into the contrast media container 30. When combined together in this way, the contrast media container 30 could be mounted directly to the pump drive system 50 or, as shown in FIG. 1, the combined battery and contrast media container 30 can be electrically coupled to the pump drive system 50 using a combined cable 35. Alternatively, the battery 20 may be sold separately from the contrast media container, but its appropriate energy capacity may be determined according to the volume of the contrast media container. In any configuration in which the battery is associated with the contrast media container 30, the battery must be placed in the non-sterile area 90. In a further exemplary embodiment, the battery 20, 120, 220 may be integrated with the pump 40, 140, 240, 340, despite providing a less certain determination of the necessary battery capacity. In such a case, the battery capacity may be determined by the maximum amount of contrast media fluid that can be safely or necessarily injected into the subject patient to perform the procedure.

The battery 20, 120, 220 may also be connected to an energy-storage capacitor, in which the battery charges the capacitor between injections. As a result, when an injection is actually taking place, the capacitor is able to provide a higher current and energy than the battery is able to on its own. By incorporating such a capacitor, a high-pressure bolus of fluid may be injected from the pump system.

Regarding the radial angiography catheters 70 for use in system 10, the catheters may be comprised of a variety of suitable materials and may take a variety of sizes and shapes. For example, the catheters may be comprised of single-wall polymers, dual-wall (stiff/flexible) polymers, or multi-wall polymer tubes that have been internally reinforced with a coil (spiral) or a braid that is comprised of metal, polymer, ceramic fiber, or a combination of these. Alternatively, the catheters may be constructed of metal tubing comprised of, for example, Nitinol (NiTi) or other tubing materials having a shape memory and super elasticity, stainless steel hypodermic tubing, aluminum tubing (which has a low stiffness for easy manipulation), and cobalt-chrome alloy, Ti-alloy, or low-modulus Zr-based alloys, including metallic glasses. A set of variably-sized, shaped and/or constructed catheters may be provided as a part of the system 10.

Also, to aid in the physician's ability to easily handle and manipulate the catheters 70, one or more of the catheters may be constructed to have a larger diameter and freedom from hydrophilic coating in the area of the catheter that is manipulated by the physician. Futhermore, the catheter may have an integrated or attachable “torquer” or holding device to facilitate twisting, pushing, or pulling of the catheter by the physician.

To connect the catheter 70 to the expansion hose or tube 65, 105, 165, 215, a special “quick connect” connector, as described above, may be used. In addition, a swivel capability may be incorporated into the connector to provide strain relief at the connection for reducing its resistance to torque and steering.

To reduce the number of wires running between the pump and the patient and/or the hand control device 80 on the injection side (i.e., distal side) of the pump, these wires can be “integrated” into the extension hose or tube 65, 105, 165, 215 by extruding the extension hose or tube so that it includes conductive wires. Or, the extension hose or tube can be extruded with lumens to accommodate wires that can be added during manufacturing.

In any of the configurations envisioned herein, including the embodiments described in detail above, a number of safety features may be implemented to ensure the safety of a patient undergoing a procedure that is performed using the hereindescribed systems and methods. For example, as described above, it is critical that air bubbles are not introduced into the catheter and do not enter the patient's bloodstream. Apart from the use of various valve mechanisms described above, an air bubble excluder system may, for example, be placed in line with the expansion hose or tube 65, 105, 165, 215, thereby creating an air-permeable “window” in the tubing set that will eliminate any air bubbles that enter the expansion hose or tube. In another example, an air bubble detector system that is operable to sense that an air bubble is present in a fluid flow of a tube and to provide an electrical signal to alert the physician of the positive presence of the air bubble, may also be placed in line at the low pressure side or the high pressure side of the pump system 110. The sensor element of this air bubble detector system may be an optical or ultrasonic device or it may be a pressure sensor that is operable to detect a change in pressure caused by the presence of air that has been inadvertently introduced into the system. The pump drive system 50, 250 of the system 10 may also be programmed and configured to have an “auto purge” function in which, once the system 10 is completely assembled but prior to introducing the catheter into the patient, upon use of a single control, the pump drive system automatically pumps the correct amount of contrast media fluid that completely purges the entire system of any air bubbles that might exist. This pre-determined purge amount of contrast media fluid may be the same for all types of catheters. Or, this pre-determined amount may be dependent upon the properties of the specific catheter that is coupled to the powered injector. As mentioned above, an authentication process may be used to determine the identity and properties of the catheter using, for example, a programmable identification chip that has been integrated into the catheter and can be authenticated by the pump drive system. However, any mechanical or electronic feature capable of providing this identification function of the catheter may be used.

Further safety features may be implemented to protect the patient and the integrity of the catheter from any damage that might be caused by injecting the contrast media fluid at too high of a pressure or too high of a flow rate for that particular patient and/or for that particular catheter. For example, a “Maximum Flow Rate” element or a “Maximum Pressure” element may be used to actively control the flow rate and/or the pressure of the injection to prevent the flow rate and/or pressure from exceeding a pre-determined level that is dependent upon the patient, catheter type, or other parameter. This element may comprise an active device (e.g., sensor/actuator) or a passive device (e.g., pressure or flow regulator).

In addition, various redundant safety controls may be implemented into the system to prevent the physician from accidentally activating the powered injector device. In one exemplary embodiment, an “arming” switch may be used that is separate from the “injection” controls and must be activated first using the control device 80 before any of the injection controls become operational. This arming switch may be implemented using an electrical switch that is present, for example, in the drive wire to the motor of the pump drive system. Or, alternatively, the arming switch may be implemented using a mechanical interlock. In another exemplary embodiment, an actual brake or any other suitable mechanical mechanism may be implemented into the pump drive system that requires manual disengagement of the brake or other mechanism by the physician before an injection can occur. In a further exemplary embodiment, a two-stage trigger may be used whereby the physician must apply a first pressure to the trigger to affirmatively initiate (or activate) the system and, thereafter, must apply a further second pressure to the trigger to cause the pump drive system to proceed with an actual injection or to regulate the pressure and/or flow rate of an actual injection.

In any of the configurations envisioned, including the embodiments described in detail herein, a number of refinements may be made to the system 10 to optimize its function. For example, with respect to the contrast media fluid, it is beneficial to increase the temperature of the fluid to lower its viscosity so that there will be less resistance to its flow through the pump system and the patient. In exemplary embodiments in which the contrast media supply 30 container is placed in close proximity or in actual contact with the battery 20, or is combined with the battery (see e.g., the embodiment of FIG. 1), heat generated by the battery may be conductively used to heat the contrast media fluid. In other exemplary embodiments, the contrast media fluid may be warmed through a heat exchange that could occur between the pump drive system and the contrast media supply 30 container, or that occurs between pump and the pump tubing while the contrast media fluid is moving therethrough. In a further exemplary embodiment, the contrast media fluid may be warmed through resistance heating of one of the hoses or tubes of the system 10 and controlling the temperature by sensor or, for example, by using an NTC resistance heater or a PTC device.

It is beneficial to synchronize injection of the power injector device with the cardiac R-wave of the patient to achieve a maximum contrast ratio and obtain a well-defined image and to reduce the amount of contrast media fluid that is used in the procedure. To accomplish this synchronization, the cardiac phase of the patient is dynamically detected. The cardiac phase may be determined by detecting the pressure pulse in the catheter 70 or in the extension hose or tube 65, 105, 165, 215. Alternatively, the cardiac phase may be detected by using a remote or wireless pulse sensor (e.g., an ultrasonic or photoplethysmography device) that is attached to the patient, or by remote detection using another monitoring instrument that is present in the operating room or catheter lab.

Various improvements can be made to the catheter introducer sheaths, as well. For example, the introducer sheath may be constructed to have a side arm connector for a heparin drip that can be used by the physician to prevent clotting in the introducer sheath after the catheter has been withdrawn. The side arm connector could also be used for the extraction of arterial blood samples for measuring arterial oxygen content. This side arm connector could be comprised of a shutoff “quick-connect”-type connector or a luer-lock-type fitting.

In addition, a grip element may be provided near or on the introducer sheath to aid the physician in handling and twisting the catheter when navigating it to the desired position in a patient. Such a grip element is particularly useful for catheters that do not have a circular, round shape (e.g., hexagonal, triangular, octagonal, grooved, etc.)

An exemplary embodiment of the system 10 incorporating some of the improvements disclosed above, with additional improvements, is illustrated in the overall view of the system 10 of FIG. 7 and with particular aspects of the system 10 being shown in FIGS. 8 to 33.

As set forth above, a mechanized syringe pump can be used for the pump 40, 140, 240, 340 and such a pump is used in the embodiment of FIG. 7. In this syringe pump 700, a sterile angiographic syringe 710 is mounted on the syringe pump 700. The piston 720 (or plunger) of the syringe 710 is connected or coupled to a pushing element 810 of the pump, which is illustrated in the views of FIGS. 8 to 12 and particularly in the views of FIGS. 10 and 11. In this embodiment, the pushing element 810 is a rack that is coupled to a motorized drive element 820 of the pump 700, which takes the form of a pinion gear.

Upon controlled actuation of a drive motor 830 of the pump 700, via a speed-controlling transmission 840, the pushing element 810 causes the piston 720 of the syringe 710 to be pulled or pushed within the cylindrical barrel of the syringe 710. Connection of the pushing element 810 to the piston 720 is carried out with an element cutout 1310 formed into the piston 720 and shown in FIG. 13 and in the transparent view of the syringe/piston in FIG. 8. The pushing element 810 is, in an exemplary embodiment, fastened into the element cutout 1310 with adhesive. Alternatively, the pushing element 810 can be an integral part of the piston 720.

When the piston 720 is pulled, a negative pressure is created in the barrel of the syringe 710 thereby drawing fluid into the barrel. When the piston 720 is pushed, a positive pressure is created in the barrel of the syringe 710 causing the fluid to be forcibly dispensed out from the barrel. Even though there are several ways in which this syringe pump 700 could be made for the high-pressure injections needed to perform radial angiography procedures, the syringe 710 shown in this exemplary embodiment is a high-strength, reusable syringe with a wall sufficient to support the high pressure needed. With the pressures used, the syringe barrel does not expand under the pressure. As shown in FIGS. 7, 8, and, in particular, FIG. 9, the piston 720 is fitted with a compliant distal seal 722.

As described above and as depicted in FIGS. 1 to 4, the pump 700 is controlled by the user with a remote hand control device 730, which is depicted and explained in further detail in FIGS. 22 to 33 below. As a result, the hand control device 730 is positioned in the sterile field and is easily accessible by the user (e.g., the physician). The remote hand control device 730 is electrically connected to the drive motor 830 through a control cord 732, which in this exemplary embodiment, is a removable control cord 732 having a easy-out disconnector 734. As such, the remote hand control device 730 and its control cord 732 can be disposed of or sterilized as desired or as designed. Thus, the hand control device 730 is allowed to easily traverse the sterile barrier with the control cord 732, thereby increasing the user's mobility. More significantly, the hand control device 730 is configured to be removably connected to a standard entry sheath 2210 used with standard angiography catheters 740 (illustrated only diagrammatically with a dashed line in FIG. 7).

Fluidic connection of the barrel of the syringe 710 to the contrast media supply 750 occurs at the distal output end of the syringe 710 at which a check valve 760 is disposed. When the pump 700 is being driven by the pump drive system 710, 720, 830, 840, contrast media fluid is drawn into the barrel of the syringe 810 through a fluid supply or media access line 752 that is connected to the contrast media supply 750. As indicated, the contrast media supply 750 includes a non-illustrated iodinated contrast media fluid housed in a specific container (e.g., a glass bottle with a fluid connection and a check valve). To accomplish filling of the syringe 710, the fluid supply line 752 is attached to the three-way manifold of the check valve 760. When the piston 720 is drawn to create a vacuum in the syringe 710, the check valve 760 allows contrast media to pass therethrough from the media access line 752 into the barrel of the syringe 710. When the piston 720 is caused to pressurize the contrast media in the syringe 710, the check valve does not permit the fluid to pass into the media access line 752 and only allows the contrast media to enter and pass through the angiography catheter 740 and into the patient. Here, the vacuum created by such withdrawal of contrast media in the contrast media supply 750 is stabilized by a one-way check valve, for example, in the lid of the supply container.

Before use, any air can be removed from the angiography catheter 740 by prefilling the catheter 740 with contrast media or an inert fluid (such as saline) or the catheter 740 can be connected to the output side of the check valve 760 and purged by discharging a small amount of contrast media through the catheter 740 and into the surgical area.

The pump 700 is supplied with electrical power from a self-contained power source 770 through one or more electrical wires or cables 772. In an exemplary embodiment, the power source 770 is a battery that is integrated into the body of the pump 700, thereby eliminating the system of a separate power supply component. The battery compartment 1810 is best seen in the hidden view of FIG. 18 and the end view of FIG. 19, in which an end cap 1710 is removed.

It is noted that precise control of the filling and ejection of contrast media in the pump 700 is dependent upon the piston 720 moving within a preset range. This control is effected by placement of two limit switches 1010, 1020 to interact with the piston 720 during its travel and placement of two switch actuators 1410, 1420 on the piston 720. As best seen in FIG. 14, the switch actuators 1410, 1420 take an exemplary form of cam surfaces that, when contacting the limit switches 1010, 1020, cause the respective limit switch 1010, 1020 to trip and stop further distal or proximal movement of the piston 720. The switch actuators 1410, 1420 can also be seen in FIG. 13 and in the hidden views of FIGS. 8 and 9. As the piston 720 retracts to fill the barrel of the syringe 710 with fluid, the fill limit actuator 1410 closes the fill limit switch 1010. Likewise, as the piston 720 extends to deplete the barrel of the syringe 710 of fluid, the empty limit actuator 1420 closes the empty limit switch 1010.

Electronic control of the drive motor 830 is carried out by a circuit board 2010 contained within the shell of the pump 700. The pump 700 can be programmed to carry out various fill and discharge routines. One exemplary discharge routine is referred to as a scout mode. In this mode, a small discharge of 1 cc, for example, is caused to occur. The scout mode is beneficial when the angiography catheter 740 is being guided to the surgical site and the surgeon desires to see where the tip of the catheter 740 resides. Even though many such puffs of contrast media can occur without fully depleting the syringe 710, the scout mode programs the pump 700 to automatically refill the syringe 710 completely after each puff occurs or shortly thereafter if two or more puffs occur within a short time frame of one another. In this way, if a large bolus of contrast media is desired, the pump 700 is ready to provide the large bolus at all times. Another exemplary discharge routine carries out a standard bolus of contrast media that is useful for standard angiography techniques. This bolus can vary, but is in the range of about 10 cc to about 20 cc.

Even though the circuit board 2010 can be programmed to adjust the speed of discharge and refill, another way to adjust the speed is through a manual dial 2110, such as a potentiometer, that can be connected to the circuit board 2010 or can be connected to the power supply directly to mete out less or more power as desired.

As indicated above, the remote hand control device 730 is disposed away from the pump 700 and desirably close to the entry point of the angiography catheter 740. This is a desirable location because the physician typically uses two hands to manipulate the angiography catheter 740, one to hold the entry sheath 2210 and the other to hold the angiography catheter 740 and move it through the entry sheath 2210 and to the surgical site to be viewed. If the control for contrast media is anywhere away from this location of the surgeon's two hands, then the surgeon must remove his/her hand to inject contrast media or another person must be directed to control the distribution of contrast media. The present invention places the control of contrast media directly at the site of the entry sheath 2210. The hand control device 730 is shaped to removably connect to the entry sheath 2210, here in a press fit having a structure that provides a form locking connection 2430 that temporarily contains the entry sheath 2210 therein. A form-locking or form-fitting connection is one that connects two elements together due to the shape of the elements themselves, as opposed to a force-locking connection, which locks the elements together by force external to the elements. The remote hand control device 730 has desirable features for use at the entry sheath 2210. First, the remote hand control device 730 has two grasping surfaces 2232, 2336 for use by a surgeon's thumb and one of the forefingers. A two-stage switch 2336 is placed on the thumb grasping surface 2334 and has a resistance that prevents entering the first pressed-in state with normal grasping and movement of the hand control device 730 by the physician. With force sufficient to enter the first pressed-in state, the scout mode is entered and a puff is carried out by the pump 700 each time this state occurs. In contrast, with force sufficient to enter the second pressed-in state, the standard bolus mode is entered and a bolus is discharged by the pump 700 to show the site under inquiry. FIGS. 25 and 26 show the two-stage switch 2336 removed from the hand control device 730 making the switch cavity 2630 visible.

FIGS. 27, 28, and 29 illustrate an alternative embodiment of the remote hand control device 730 where the two-stage switch is separated into two separate switches 2710, 2720 and these switches 2710, 2720 are located on the forefinger side of the remote hand control device 730. FIGS. 30 to 33 illustrate yet another alternative embodiment of the remote hand control device 730 where two separate switches 3010, 3020 are located likewise on the forefinger side of the remote hand control device 730.

As such, the system 700 comprises the pump, pump drive system, and power supply that are all combined or integrated into a single, self-contained unit. By combining or integrating all of these components into a single unit, it is relatively compact and lightweight to be easily portable and easily transported into an area adjacent to or shielded from the sterile field.

It is noted that various individual features of the inventive processes and systems may be described only in one exemplary embodiment herein. The particular choice for description herein with regard to a single exemplary embodiment is not to be taken as a limitation that the particular feature is only applicable to the embodiment in which it is described. All features described herein are equally applicable to, additive, or interchangeable with any or all of the other exemplary embodiments described herein and in any combination or grouping or arrangement. In particular, use of a single reference numeral herein to illustrate, define, or describe a particular feature does not mean that the feature cannot be associated or equated to another feature in another drawing figure or description. Further, where two or more reference numerals are used in the figures or in the drawings, this should not be construed as being limited to only those embodiments or features, they are equally applicable to similar features or not a reference numeral is used or another reference numeral is omitted.

The phrase “at least one of A and B” is used herein and/or in the following claims, where A and B are variables indicating a particular object or attribute. When used, this phrase is intended to and is hereby defined as a choice of A or B or both A and B, which is similar to the phrase “and/or”. Where more than two variables are present in such a phrase, this phrase is hereby defined as including only one of the variables, any one of the variables, any combination of any of the variables, and all of the variables.

The foregoing description and accompanying drawings illustrate the principles, exemplary embodiments, and modes of operation of the invention. However, the invention should not be construed as being limited to the particular embodiments discussed above. Additional variations of the embodiments discussed above will be appreciated by those skilled in the art and the above-described embodiments should be regarded as illustrative rather than restrictive. Accordingly, it should be appreciated that variations to those embodiments can be made by those skilled in the art without departing from the scope of the invention as defined by the following claims.

What is claimed is:

1. An integrated catheter and powered injector system, comprising:
a pump system housed in a self-contained unit, the pump system comprising:
a motorized pump drive sub-system;
a pump electrically connected to and operated by the pump drive sub-system, the pump having a input and an output; and
a battery electrically connected to and powering the pump drive sub-system;
a contrast media supply removably connected to the pump system by at least one fluid supply line and containing contrast media fluid;
an extension line shaped to removably connect an arterial catheter to the pump system; and
a control device operatively connected to the pump system to allow a user to send control signals to the pump system such that, in response to control signals received from the control device, the pump drive sub-system drives the pump to:
draw the contrast media fluid from the contrast media supply into the input of the pump through the at least one fluid supply line; and
inject the contrast media fluid from the output of the pump through the extension line and into the arterial catheter at a controlled flow rate and/or pressure.

2. The system according to claim 1, wherein the battery is removable.

3. An integrated catheter and powered injector system, comprising:
a battery-powered pump drive system;
a peristaltic pump electrically connected to and operated by the pump drive system, the peristaltic pump having an input and an output;
a removable battery electrically connected to and powering the pump drive system;
a contrast media supply removably connected to the input of the peristaltic pump by at least one fluid supply line;
an extension line shaped to removably connect an arterial catheter to the output of the peristaltic pump; and
a control device operatively connected to the pump drive system to allow a user to send control signals to the pump drive system such that, in response to control signals received from the control device, the pump drive system drives the peristaltic pump to:
draw contrast media fluid from the contrast media supply into the input of the peristaltic pump through the at least one fluid supply line; and
inject the contrast media fluid from the output of the peristaltic pump through the extension line and into the arterial catheter at a controlled flow rate and/or pressure.
